(19) United States
(12) Patent Application Publication
(10) Pub. No.: US 2006/0263429 A1
(43) Pub. Date: Nov. 23, 2006

(54) Compressible mixture, compressed pharmaceutical compositions, and method of preparation thereof

(76) Inventor: Hengsheng Feng, Edison, NJ (US)

Correspondence Address: CANTOR COLBURN, LLP, 55 GRIFFIN ROAD SOUTH, BLOOMFIELD, CT, 06002, US

(21) Appl. No.: 11/133,864

(22) Filed: May 20, 2005

(51) Int. Cl. A61K 9/26

(52) U.S. Cl. 424/469

(57) ABSTRACT

A compressible mixture prepared from a waxy filler, cellulose filler, or a mixture thereof and a disintegrant is disclosed for the preparation of compressed pharmaceutical compositions containing coated pellets. The resulting compressed compositions exhibit substantially the same dissolution profiles as the pellets in the absence of the compressible mixture.

### BACKGROUND OF THE INVENTION

Modified- or controlled-release pharmaceutical dosage forms provide the advantages of releasing a pharmaceutical agent to a patient in a controlled manner. Controlled-release may include sustained-, delayed- or pulsed-release at a particular time. Alternatively, controlled may mean extended-release of the pharmaceutical agent allowing for the reduction in the number of doses the patient is administered in a given timeframe. Such controlled-release can prevent “dose dumping”, extreme fluctuations in the plasma concentration of the pharmaceutical agent, and the like.

Compared to a conventional tablet, pellets in tablets provide the following benefits: the ability to carry a large dose of pharmaceutical agent, the ability to obtain a wide range of release profiles, a reduction in dose to dose variability, more reliable dose to dose release, and a reduction in bio-variability. Pellets allow for ease and convenience of manufacturing as they can be further processed into a capsule or a tablet.

To prepare solid dosage forms, compression is the most favorable process. Despite its advantages, such as reduced processing time and cost, the success of compression is determined by the chemical and physical properties of the mass carrying the pharmaceutical agent, choice of fillers, behavior of the mixture during process and the desired properties of the final dosage form (e.g. release properties). The difficulty in formulation is increased when the number of pharmaceutical agents is increased, the form of the pharmaceutical agent (e.g. powder versus granules, pellets, beads, etc.), whether the pharmaceutical agent is coated or formulated to have a particular size, shape, or other property.

The following literature discusses the challenges of preparing compression dosage forms from coated granules or discrete units of pharmaceutical agents:

U.S. Pat. No. 4,874,614 generally discloses a method of preventing the fracture of coated drug granules during a compression of the granules into a tablet matrix, wherein the drug release of the tablet decreases with time while increasing the amount of microcrystalline cellulose in the dosage form.

U.S. Pat. No. 5,780,055 generally discloses “cushioning beads” prepared from microcrystalline cellulose and a disintegrant such as croscarmellose sodium. The cushioning beads are prepared to be mechanically weaker than the coated beads containing active ingredients. The cushioning beads cushion the coated beads from damage when compacted. The disclosed beads had an average diameter of about 0.2-2.0 mm and preferably about 0.5-1.5 mm.

GB 1 598 458 generally discloses compression tableting of microcapsules with 2-20% w/w of a water-soluble wax.

EP 1 131 057 generally discloses cushioning beads made from a microcrystalline hydrocarbon wax or a natural wax in an amount of at least 30% by weight of the cushioning beads. The beads are used to prepare solid shaped articles containing biologically active ingredients by compression. To minimize the occurrence of segregation between active ingredient-loaded pellets and the cushioning beads, the '057 patent discloses that the inert beads should be of the same size and approximately the same density as the active pellets. The cushioning beads are described by an average particle size of about 0.5 to about 2.0 mm and most preferably from 0.75 to 1.25 mm. Furthermore, the '057 patent distinguishes beads or pellets from granules, as pelletization is an agglomeration process that converts fine powders or granules into small, free-flowing, spherical or semi-spherical units. It is additionally stated that, as opposed to the process of granulation, the production of beads results in a narrow size-range distribution.

### BRIEF SUMMARY OF THE INVENTION

Disclosed herein are compressed pharmaceutical compositions comprising a plurality of coated pellets, wherein the coated pellets comprise a pharmaceutically active agent or salt, solvate, hydrate, or polymorph thereof; and a compressible mixture, wherein the compressible mixture comprises i) a non-water soluble waxy filler and ii) a disintegrant, and wherein the compressible mixture is in powder form, with the proviso that the compressible mixture is not prepared by extrusion, spheronization, high shear mixing, melt blending, melt pelletization, freeze-drying, or a combination thereof, and wherein the compressed composition exhibits an in vitro dissolution profile according to a USP compendia method that is substantially the same as the dissolution of the coated pellets in the absence of the compressible mixture.

In another embodiment, a process of making a compressed pharmaceutical composition comprises mixing a powdered, waxy filler and a disintegrant to form a compressible mixture, wherein the waxy filler is non-water soluble; mixing the compressible mixture with a plurality of coated pellets to form a pellet mixture, wherein the coated pellets comprise a pharmaceutically active agent or salt, solvate, hydrate, or polymorph thereof; and compressing the pellet mixture with conventional direct compression equipment, wherein the compression force is not more than about 25 kiloNewtons.

In another embodiment, a direct compression compressible mixture for forming compressed pharmaceutical compositions comprises a mixture of a powdered waxy filler and a disintegrant, optionally further comprising a cellulose filler, a binder, a lubricant, a glidant, a compression aid, a colorant, a preservative, a flavor, or combinations thereof, wherein the mixture has an average particle diameter of about 0.1 to about 125 micrometers, with the proviso that the compressible mixture is not prepared by extrusion, spheronization, high shear mixing, melt blending, melt pelletization, freeze-drying, or a combination thereof; and wherein the compressible mixture is suitable for directly compressing mixtures of the compressible mixture and coated drug pellets with substantially no damage to the coated drug pellets.

### BRIEF DESCRIPTION OF THE DRAWINGS

FIG. 1. Dissolution comparison for diltiazem HCl tablets and pellets

FIG. 2. Dissolution comparison for diltiazem HCl tablets and pellets

FIG. 3. Dissolution comparison for morphine sulfate tablets and pellets

FIG. 4. Dissolution comparison for amphetamine tablets and pellets

FIG. 5. Dissolution comparison for diltiazem HCl pellets and tablets prepared from wax and microcrystalline cellulose,

### DETAILED DESCRIPTION OF THE INVENTION

Disclosed herein are compressible mixtures which, when directly compressed with coated drug pellets, provide excellent protection of the pellet and pellet coating while at the same time providing a compressed pharmaceutical composition (e.g., tablet) having adequate hardness and low friability. As a result of the minimal damage to the pellet or pellet coating and the particular choice of mixture components, the resulting compressed pharmaceutical compositions exhibit a dissolution profile substantially the same as the dissolution profile of the coated drug pellets alone. Furthermore, the compressible mixtures blended with drug pellets can be used with conventional tableting equipment and require no special tools or machinery.

When the compressible mixture is prepared from a waxy filler, segregation is not observed between the drug pellets and the compressible mixture even when the waxy filler is in powdered form.

Also disclosed herein are compressed pharmaceutical compositions prepared from direct compression of a blend of compressible mixture and drug pellets. Methods of tableting the blend are also provided.

As used herein a “drug pellet” means subunits of a dosage form comprising an active agent in combination with a binder and other optional inert ingredients that have been formed into particles, pellets, granules, spheres or spheroids, beads, etc., (collectively referred to herein as “pellets”).

As used herein a “coated pellet” or “coated drug pellet” means a pellet as defined above, which has been further coated with a functional or non-functional coating as described herein.

The compressible mixture used, to prepare compressed pharmaceutical compositions containing drug pellets comprises a compression filler which is a waxy filler, a cellulose filler, or a mixture thereof and a disintegrant, specifically a “super” disintegrant. The compressible mixture is provided as a powdery or particulate material and is not further processed or compressed prior to combining with a pellet and subsequent compression into a compressed pharmaceutical composition.

As used herein, a “tablet”, “compressed tablet”, or “compressed pharmaceutical composition” mean the same unless otherwise indicated.

The compression filler (waxy filler, cellulose filler or a mixture thereof) provides a variety of functions. First, it protects the coated pellets during the compression process by absorbing the compressive forces thereby relieving or removing compressive force on the coated pellets. The resulting compressed pharmaceutical composition exhibits minimal damage to the pellets. Second, it acts as a binder to hold the pellets together once compressed. The resulting compressed pharmaceutical composition exhibits excellent hardness and minimal friability even in the absence of additional binders.

Exemplary waxy fillers include waxes such as carnauba wax (from the palm tree Copernicia Cerifera), vegetable wax, fruit wax, microcrystalline wax (“petroleum wax”), bees wax (white or bleached, and yellow), hydrocarbon wax, paraffin wax, cetyl esters wax, non-ionic emulsifying wax, anionic emulsifying wax, candelilla wax, combinations thereof, and the like. Other suitable waxy fillers include, for example, stearyl alcohol, cetyl alcohol, cetostearyl alcohol, polyethylene glycol (PEG) having a molecular weight of greater than about 3000 number average molecular weight, Mₙ, (e.g. PEG 3350, PEG 4000, PEG 4600, PEG 6000, and PEG 8000). Each wax described herein can be in powder or flake form.

The waxy filler can specifically be a solid, hydrophobic material (i.e non-water soluble) or solid hydrophilic material (e.g. polyethylene glycols described above which are water soluble), but specifically a solid, hydrophobic material in powder form.

The waxy material can be in the form of a powder or flake. When in the form of a powder, the waxy material can have an average particle diameter of up to about 175 micrometers, specifically an average particle diameter of about 0.1 micrometers to about 150 micrometers, more specifically about 1.0 micrometers to about 100 micrometers, and yet more specifically about 10 to about 75 micrometers. When in the form of flakes, the waxy material can be milled into desired sizes and sieved using mesh filters. When milled, the waxy material can be maintained at a temperature and/or at low shear to prevent melting and agglomeration.

The melting point of the waxy material may be at any temperature above room temperature, specifically about 30 to about 150° C., more specifically about 75 to about 100° C., and yet more specifically about 75 to about 90° C.

The cellulose filler can be any cellulose material that can provide a direct compressed pharmaceutical composition containing coated pellets where there is substantially no damage to the pellets or pellet coating. Exemplary cellulose fillers include powdered cellulose having a “cottony” or “fluffy” characteristic (non-flowing), or microcrystalline cellulose (Avicel PH microcrystalline celluoses, e.g. Avicel PH-102).

The amount of waxy filler, cellulose filler or a combination thereof that can be used in the compressed pharmaceutical composition (total weight of the drug pellets and compressible mixture) can be up to about 75 weight percent based on the total amount of the composition, specifically about 10 to 70 weight percent, more specifically about 20 to about 60 weight percent, and yet more specifically about 30 to about 50 weight percent based on the total amount of the compressed pharmaceutical composition.

The disintegrant present in the compressible mixture is used to facilitate the breakdown of the compressed or compacted compressible mixture in a fluid environment, specifically aqueous environments. The choice and amount of disintegrant can be tailored to ensure the dissolution profile of the tablet is substantially the same as the dissolution profile of the drug pellets alone. In an alternative embodiment, the choice and amount of disintegrant is tailored to provide additional release-retarding properties for those formulations where additional controlled-release is desired.

The disintegrants used in combination with the waxy filler or cellulose filler to form the compressible mixture include so-called “super” disintegrants known in the art. Exemplary super disintegrants include, for example, cross-linked sodium carboxymethylcellulose (“croscarmellose sodium”, i.e., Ac-Di-Sol® available from FMC BioPolymer of Philadelphia, Pa.); crosslinked homopolymer of N-vinyl-2-pyrrolidone (“crospovidone”, e.g., Polyplasdone® XL, Polyplasdone® XL-10, and Polyplasdone® INF-10 available from International Specialty Products, Wayne N.J.); modified starches, such as sodium carboxymethyl starch, sodium starch glycolate (e.g., Primogel), and the like; alginates; and combinations thereof.

The amount of disintegrant used can be about 1 to about 10 weight percent based on the total weight of the compressed pharmaceutical composition, specifically about 2 to about 7 weight percent, and yet more specifically about 3 to about 5 weight percent. The ratio of compression filler to disintegrant can be about 15:1 to about 50:1, specifically about 20:1 to about 45:1, and yet more specifically about 25:1 to about 40:1.

In addition to the super disintegrants, additional disintegrants that generally possess the ability to swell or expand upon exposure to the fluid environment, especially an aqueous environment can be used alone or in combination with the super disintegrants. Examples of such disintegrants are starch, and pregelatinized starch (e.g., Starch 1500® available from Colorcon).

In another embodiment, a combination of waxy and cellulose fillers can be used to allow for the tailoring of the resulting compressed pharmaceutical composition properties. For example, the compressible mixture containing only a waxy filler as the compression filler, provides a compressed pharmaceutical composition that is easily broken by hand (i.e. with finger pressure) without the presence of a score line on the compressed pharmaceutical composition. Such a compressed pharmaceutical composition still exhibits adequate hardness and can be packaged in bottles or in blister packs, and the like. Optionally, a coating can be provided if more protection to the compressed pharmaceutical composition is desired. To provide a harder compressed pharmaceutical composition while not damaging the coated drug pellets during compression varying amounts of a cellulose filler (e.g. microcrystalline cellulose) can be added to the compressible mixture in place of a portion of the waxy filler. The resulting compressed pharmaceutical composition is harder and less friable.

When the compression filler is a combination of a waxy filler and a cellulose filler, the ratio of the two components can be about (weight/weight) 100:0 to about 0:100; about 95:5 to about 5:95; about 90:10 to about 10:90; about 85:15 to about 15:85; about 80:20 to about 20:80; about 75:25 to about 25:75; about 70:30 to about 30:70; about 60:40 to about 40: 60; or about 45:65 to about 65:45.

Optionally, the compressible mixture can further comprise additional pharmaceutically acceptable excipients so long as the excipients do not result in significant damage the drug pellet coatings when compressed to form a compressed pharmaceutical composition. The optional additional excipients can provide desired properties, such as increased hardness of the resulting tablet, ease of manufacture of the tablet, enhancement of the taste and visual aspects of the tablet, enhanced stability, enhanced patient acceptability, etc. Pharmaceutical excipients include binders, lubricants, glidants, compression aids, colorants, preservatives, flavors, etc.

Additional optional excipients include, for example, silicified microcrystalline cellulose, powdered cellulose, polyvinyl pyrrolidone, hydroxypropyl cellulose, hydroxypropyl methylcellulose, methylcellulose hydroxyethyl cellulose, mannitol, sorbitol, lactose, digestible sugars, sucrose, liquid glucose, sorbitol, dextrose, isomalt, liquid maltitol, aspartame, lactose, talc, and the like, and combinations thereof.

The compressible mixture can further comprise a lubricant and/or glidant to aid in the tableting process. Exemplary lubricants include stearic acid, stearates (e.g., calcium stearate, magnesium stearate, and zinc stearate), sodium stearyl fumarate, glycerol behenate, mineral oil, polyethylene glycol, talc, vegetable oil, and combinations thereof. Glidants include, for example, silicon dioxide (e.g. fumed or colloidal). Certain materials can function both as a glidant and a lubricant.

The lubricant or glidant can be used in amounts of about 0.1 to about 15 weight percent of the total weight of the compressed pharmaceutical composition; specifically about 0.5 to about 5 weight percent; and yet more specifically about 0.75 to about 3 weight percent.

The colorants can include pharmaceutically acceptable dyes, pigments, and the like. Exemplary colorants include FD&C blues, greens, oranges, reds, yellows, lakes and the like; D&C blues, greens, oranges, reds, yellows, lakes, and the like; titanium dioxide; combinations thereof; and the like.

Typically the compressible mixture is prepared by merely mixing the waxy filler or cellulose filler in powder form with the disintegrant and optional additives. Care is taken to maintain the powder form of the waxy filler by keeping the mixture below the melting temperature of the waxy filler and by minimizing the shear on the mixture. The compressible mixture is thereby provided in powder form having average particle diameters of up to about 175 micrometers, specifically of about 0.1 micrometer to about 150 micrometers, more specifically about 1.0 micrometer to about 100 micrometers, and more specifically about 10 micrometers to about 75 micrometers. The compressible mixture is not formed by any of the following processes: extrusion, spheronization, high shear mixing, melt blending, melt pelletization, freeze-drying, or any combination thereof; nor is the compressible mixture prepared into beads.

It was unexpectedly discovered that the powdery compressible mixture containing the waxy filler in quantities as provided herein is able to provide direct compressed pharmaceutical compositions containing coated pellets where the pellets remain substantially undamaged. Furthermore, the resulting compressed pharmaceutical composition exhibits substantially no change in the dissolution characteristics as compared to the drug coated pellets alone. Such a result was highly unexpected as it is known that so-called “cushioning beads” prepared from wax can provide some protection to coated pellets during a direct compression tableting process. It has been previously disclosed that in order to avoid segregation between the cushioning beads and the drug pellets, that the beads and pellets should be about the same size. It was unexpectedly found that the compressible mixture prepared from a powdered waxy filler and disintegrant does not segregate when tableted in conventional tableting equipment. Not wishing to be bound by theory, but it is believed that the powdered waxy filler does not have good flowing properties which in effect retards segregation of the compressible mixture and the drug pellets.

The drug pellet can be prepared from any known pharmaceutically active agent, vitamin, dietary supplement, and the like, and is not limited thereby. The term “drug” or “active agent” is meant to include solvates (including hydrates) of the free compound or salt, crystalline and non-crystalline forms, as well as various polymorphs. Unless otherwise specified, the term “active agent” is used herein to indicate the active agent or a pharmaceutically acceptable salt thereof including any and all optical isomers, either alone or in combination.

Classes of pharmaceutically active agents that can be used in the pellets include, for example, alpha-2 adrenergic agents, analgesics, angiotensin-converting enzyme (ACE) inhibitors, antianxiety agents, antiarrhythmics, antibacterials, antibiotics, antidepressants, antidiabetics, antiemetics, antiepileptics, antifungal antihelminthics, antihistamines, antihyperlipidemics, antihypertensive agents, antiinfectives, antimalarials, antimicrobials, antimigraine agents, antimuscarinic agents, antineoplastic agents, antiprotozoal agents, antipsychotic agents, antispasmodics, antiviral agents, attention-deficit hyperactivity disorder (ADHD) agents, β-blockers, calcium channel blockers, chemotherapeutic agents, cholinesterase inhibitors, Cox-2 inhibitors, decongestants, diuretics, histamine-2 receptor antagonists, hypnotics, hypotensive agents, immunosuppresants, lipotropics, neuroleptics, opioid analgesics, peripheral vasodilators/vasoconstrictors, sedatives, serotonin receptor agonists, and the like.

Exemplary pharmaceutically active agents include amphetamine, dextroamphetamine, diltiazem, fluvastatin, hydromorphone, morphine, oxybutynin, oxycodone, paroxetine, propranolol, tolterodine, venlafaxine, their pharmaceutically acceptable salts, solvates, hydrates, and polymorphs.

In one embodiment, the coated drug pellets are the diltiazem pellets disclosed in U.S. Pat. No. 4,894,240 to Geoghegan et al., U.S. Pat. No. 5,002,776 to Geoghegan et al., U.S. Pat. No. 5,286,497 to Hendrickson et al., U.S. Pat. No. 5,364,620 to Geoghegan et al., U.S. Pat. No.5,439,689 to Hendrickson et al., U.S. Pat. No. 5,470,584 to Hendrickson et al., U.S. Pat. No. 6,214,385 to Heinicke et al., U.S. Pat. No. 6,033,687 to Heinicke et al. and U.S. Pat. No. 5,834,024 to Heinicke et al. the teachings of which are incorporated herein by reference.

In another embodiment, the coated drug pellets are the oxbutynin drug releasing beads disclosed in U.S. Pat. No. 6,262,115 to Guittard et al., the teachings of which are incorporated herein by reference.

In yet another embodiment, the coated drug pellets are the sustained-release morphine particles disclosed in U.S. Pat. No. 6,066,339 to Stark et al. and the pellets of morphine disclosed in U.S. Pat. Nos. 5,202,128 and 5,378,474 to Morella et al., the teachings of which are incorporated herein by reference.

In one embodiment, the drug pellets and coated drug pellets are the fluvastatin pellets disclosed in U.S. Pat. No. 5,356,896 to Kabadi et al. and the particles disclosed in U.S. Pat. No. 6,242,003 to Kalb et al, the teachings of which are incorporated herein by reference.

In another embodiment, the drug pellets and coated drug pellets are the oxycodone pellets disclosed in U.S. Pat. No. 5,266,331 to Oshlack et al, the teachings of which are incorporated herein by reference.

In another embodiment, the drug pellets and coated drug pellets are the venlafaxine spheroids disclosed in U.S. Pat. No. 6,274,171 to Sherman et al., U.S. Pat. No. 6,403,120 to Sherman et al., and U.S. Pat. No. 6,419,958 to Sherman et al., the teachings of which are incorporated herein by reference.

In still another embodiment, the drug pellets and drug coated pellets are the tolterodine beads disclosed in U.S. Pat. No. 6,630,162 to Nilvebrant, and U.S. Pat. No. 6,770,295 to Kreilgard et al., the teachings of which are incorporated herein by reference.

In one embodiment, the drug pellets and drug coated pellets are the opioid analgesic (e.g. hydromorphone) multiparticulates disclosed in U.S. Pat. No. 5,958,452 to Oshlack et al., U.S. Pat. No. 5,965,161 to Oshlack et al., U.S. Pat. No. 5,968,551 to Oshlack et al., U.S. Pat. No. 6,294,195 to Oshlack et al., U.S. Pat. No. 6,335,033 to Oshlack et al., U.S. Pat. No. 6,706,281 to Oshlack et al., U.S. Pat. No. 6,743,442 to Oshlack et al., and U.S. Pat. No. 6,066,339 to Stark et al. the teachings of which are incorporated herein by reference.

In still another embodiment, the drug pellets and drug coated pellets are the amphetamine beads disclosed in U.S. Pat. Nos. 6,322,819 and 6,605,300 both to Burnside et al., the teachings of which are incorporated herein by reference.

The drug pellets can be prepared by any known method in the art including melt pelletization techniques, extrusion with optional further shape modification of the resulting pellet, spheronization techniques, and the like.

The drug pellets can be coated to form coated drug pellets. The coating can be a functional or a non-functional coating, or multiple functional and/or non-functional coatings. By “functional coating” is meant to include a coating that modifies the release properties of the total formulation, for example, a sustained-release coating, extended-release coating, delayed-release coating, and the like. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated form.

The coating material may include a polymer, for example, alkyl cellulose (e.g., methyl cellulose, ethyl cellulose, and the like), hydroxypropyl cellulose, hydroxypropyl methyl cellulose, hydroxybutyl methyl cellulose, cellulose acetate, cellulose propionate, cellulose acetate propionate, cellulose acetate butyrate, cellulose acetate phthalate, carboxymethyl cellulose, cellulose triacetate, cellulose sulphate sodium salt, poly(methyl methacrylate), poly (ethyl methacrylate), poly (butyl methacrylate), poly (isobutyl methacrylate), poly (hexyl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene)high density, (poly propylene), poly (ethylene glycol), poly (ethylene oxide), poly (ethylene terephthalate), poly(vinyl alcohol), poly(vinyl isobutyl ether), poly(vinyl acetate), poly (vinyl chloride), polyvinyl pyrrolidone, and combinations thereof.

The coating may also contain an effective amount of a plasticizer in the coating composition to improve the physical properties of the film. For example, because ethyl cellulose has a relatively high glass transition temperature and does not form flexible films under normal coating conditions, it may be advantageous to add plasticizer to the ethyl cellulose before using the same as a coating material. Generally, the amount of plasticizer included in a coating solution is based on the concentration of the polymer, e.g., most often from about 1 weight percent to about 50 weight percent of the polymer. Concentrations of the plasticizer, however, can be determined by routine experimentation.

Exemplary plasticizers for ethyl cellulose and other celluloses include dibutyl sebacate, diethyl phthalate, triethyl citrate, tributyl citrate, triacetin, and combinations thereof, although it is possible that other water-insoluble plasticizers (such as acetylated monoglycerides, phthalate esters, castor oil, etc.) can be used.

Exemplary plasticizers for acrylic polymers include citric acid esters such as triethyl citrate NF, tributyl citrate, dibutyl phthalate, 1,2-propylene glycol, polyethylene glycols, propylene glycol, diethyl phthalate, castor oil, triacetin, and combinations thereof, although it is possible that other plasticizers (such as acetylated monoglycerides, phthalate esters, castor oil, etc.) can be used.

An example of a functional coating comprises a coating agent comprising a poorly-water-permeable component (a) such as, an alkyl cellulose, for example an ethylcellulose, such as AQUACOAT (a 30% dispersion available from FMC, Philadelphia, Pa.) or SURELEASE (a 25% dispersion available from Colorcon, West Point, Pa.) and a water-soluble component (b), e.g., an agent that can form channels through the poorly-water-permeable component upon the hydration or dissolution of the soluble component. Specifically, the water-soluble component is a low molecular weight, polymeric material, e.g., a hydroxyalkylcellulose, hydroxyalkyl(alkylcellulose), and carboxymethylcellulose, or salts thereof. Particular examples of these water soluble polymeric materials include hydroxyethylcellulose, hydroxypropylcellulose, hydroxyethylmethylcellulose, hydroxypropylmethylcellulose, carboxymethylcellulose, sodium carboxymethylcellulose, and combinations comprising one or more of the foregoing materials. The water-soluble component can comprise hydroxypropylmethylcellulose, such as METHOCEL (Dow). The water-soluble component is of relatively low molecular weight, specifically less than or equal to about 25,000 molecular weight, or specifically less than or equal to about 21,000 molecular weight.

In the functional coating, the total of the water soluble portion (b) and poorly-water permeable portion (a) are present in weight ratios (b):(a) of about 1:4 to about 2:1, specifically about 1:2 to about 1:1, and more specifically in a ratio of about 2:3. Other ratios can be used to modify the speed with which the coating permits release of the active agent.

Exemplary delayed-release coatings include enteric coatings prepared from enteric polymers. The enteric polymer should be non-toxic and is predominantly soluble in the intestinal fluid, but substantially insoluble in the gastric juices. Examples include polyvinyl acetate phthalate (PVAP), hydroxypropylmethyl-cellulose acetate succinate (HPMCAS), cellulose acetate phthalate (CAP), methacrylic acid copolymer, hydroxy propyl methylcellulose succinate, cellulose acetate succinate, cellulose acetate hexahydrophthalate, hydroxypropyl methylcellulose hexahydrophthalate, hydroxypropyl methylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer (acid number 300 to 330 and also known as EUDRAGIT L), which is an anionic copolymer based on methacrylate and available as a powder (also known as methacrylic acid copolymer, type A NF, methacrylic acid-methyl methacrylate copolymer, ethyl methacrylate-methylmethacrylate-chlorotrimethylammonium ethyl methacrylate copolymer, and the like, and combinations comprising one or more of the foregoing enteric polymers. Other examples include natural resins, such as shellac, SANDARAC, copal collophorium, and combinations comprising one or more of the foregoing polymers. Yet other examples of enteric polymers include synthetic resin bearing carboxyl groups. The methacrylic acid: acrylic acid ethyl ester 1:1 copolymer solid substance of the acrylic dispersion sold under the trade designation “EUDRAGIT L-100-55” may be suitable.

Suitable methods known in the art can be used to apply the coating to the drug pellet. Processes such as simple or complex coacervation, interfacial polymerization, liquid drying, thermal and ionic gelation, spray drying, spray chilling, fluidized bed coating, pan coating, electrostatic deposition, may be used. A substantially continuous nature of the coating may be achieved, for example, by spray drying from a suspension or dispersion of the drug pellets in a solution of the coating composition including a polymer in a solvent in a drying gas having a low dew point.

When a solvent is used to apply the coating, the solvent is specifically an organic solvent that constitutes a good solvent for the coating material, but is substantially a non-solvent or poor solvent for of the drug pellet. The solvent may be selected from alcohols such as methanol, ethanol, halogenated hydrocarbons such as dichloromethane (methylene chloride), hydrocarbons such as cyclohexane, and combinations comprising one or more of the foregoing solvents. Dichloromethane (methylene chloride) has been found to be particularly suitable.

The functional coating may comprise about 1 weight percent to about 40 weight percent, specifically about 3 weight percent to about 30 weight percent, more specifically about 5 weight percent to about 25 weight percent, and yet more specifically about 6 weight percent to about 10 weight percent of the total pellet weight.

The drug pellets and drug coated pellets are not limited in size. The drug pellets or drug coated pellets can have an average diameter of about 100 micrometers or greater, specifically about 100 to about 3000 micrometers, more specifically about 500 to about 1800 micrometers, and yet more specifically about 700 to about 1200 micrometers.

The amount of coated drug pellet in the compressed pharmaceutical composition can be up to about 70 percent by weight based on the total tablet. When the integrity of the pellet and pellet coating is desired, lower amounts are suggested due to the potential damage caused by pellet to pellet interaction at high loadings. Specifically the amount of coated drug pellet can be about 10 to about 60 weight percent, more specifically about 20 to about 50 weight percent, and yet more specifically about 30 to about 40 weight percent based on the total weight of the compressed pharmaceutical composition.

Another aspect is to provide a process to make compressed pharmaceutical compositions containing coated drug pellets. The compressible mixtures as disclosed herein, are particularly suited for direct compression processes even with drug pellets coated with material that is considered inelastic and easily subject to damage when compressed (e.g. ethyl cellulose). Direct compression, using commercially available punches and dies fitted to a suitable rotary tableting press, can be used. In common tableting processes, the material that is to be tableted is deposited into a cavity, and one or more punch members are then advanced into the cavity and brought into intimate contact with the material to be pressed, whereupon compressive force is applied. The material is thus forced into conformity with the shape of the punches and the cavity.

The compression force that is suitable for preparing the compressed pharmaceutical compositions via direct compression can be about 2 to about 25 kiloNewtons (kN), specifically about 3 to about 20 kN, and more specifically about 5 to about 15 kN. It has been found that within these ranges, the tableting force used has little effect on the dissolution profile of the resulting compressed pharmaceutical composition.

The compressed pharmaceutical compositions formed exhibit a hardness of at least about 5 kilopond (kp), specifically at least about 8 kp; more specifically at least about 10 kp; and yet more specifically at least about 12 kp. Direct compression techniques are preferred for the formation of the compressed pharmaceutical compositions. When compressed pharmaceutical compositions are made by direct compression, the addition of lubricants may be helpful to promote powder flow and to prevent capping of the particle (breaking off of a portion of the particle) when the pressure is relieved.

The compressed pharmaceutical compositions do not have to undergo post-compression processes such as sintering or coating as the compressed compositions exhibit suitable hardness and friability characteristics. However, if desired the tablets may optionally be further coated with a non-functional or functional coating as described above. Additionally, the compressed compositions can undergo a sintering process to meld the surface of the composition.

The compressed pharmaceutical composition can be prepared in a variety of geometrical shapes and sizes by use of different punches and dies. The compressed pharmaceutical composition can be compressed into scored forms (e.g. one score line to allow for the tablet to be split into two; or two score lines to allow the tablet to be split into three pieces) or, as discussed previously, compressed compositions free of a score.

In one embodiment, a compressed pharmaceutical composition is formed by the process comprising mixing a powdered non-water soluble waxy filler and a disintegrant to form a compressible mixture having average particle diameters of about 0.1 to about 125 micrometers, with the proviso that the compressible mixture is not prepared by extrusion, spheronization, high shear mixing, melt blending, melt pelletization, freeze-drying, or a combination thereof; mixing the compressible mixture with a plurality of coated pellets to form a compressible mixture, wherein the coated pellets comprise a pharmaceutically active agent or salt, solvate, hydrate, or polymorph thereof; and directly compressing the compressible mixture into a compressed pharmaceutical composition.

The drug pellets, coated drug pellets, and the compressed pharmaceutical compositions prepared from compressing a mixture of pellets and compressible mixture can be characterized by their dissolution profiles. Dissolution profile as used herein, means a plot of the cumulative amount of active ingredient released as a function of time. The dissolution profile can be measured utilizing the Drug Release Test <724>, which incorporates standard test USP 28 (Test <711>). A profile is characterized by the test conditions selected. Thus the dissolution profile can be generated at a pre-selected apparatus type, shaft speed, temperature, volume, and pH of the dissolution media.

A first dissolution profile can be measured at a pH level approximating that of the stomach. A second dissolution profile can be measured at a pH level approximating that of one point in the intestine or several pH levels approximating multiple points in the intestine.

A highly acidic pH may simulate the stomach and a less acidic to basic pH may simulate the intestine. By the term “highly acidic pH”: it is meant a pH of about 1 to about 4. By the term “less acidic to basic pH” is meant a pH of greater than about 4 to about 7.5, specifically about 6 to about 7.5. A pH of about 1.2 can be used to simulate the pH of the stomach. A pH of about 6 to about 7.5, specifically about 6.8, can be used to simulate the pH of the intestine. Exemplary dissolution media include 500 or 900 ml of purified water; an aqueous buffer solution (USP, pH 4.5); an aqueous buffer solution (USP, pH 6.8); an aqueous buffer solution (USP, pH 7.5); or 0.1N HCl.

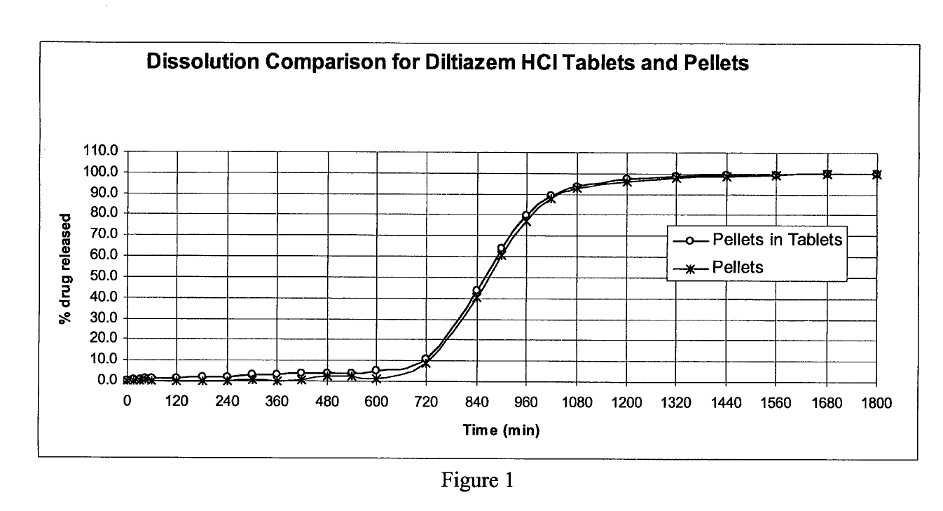

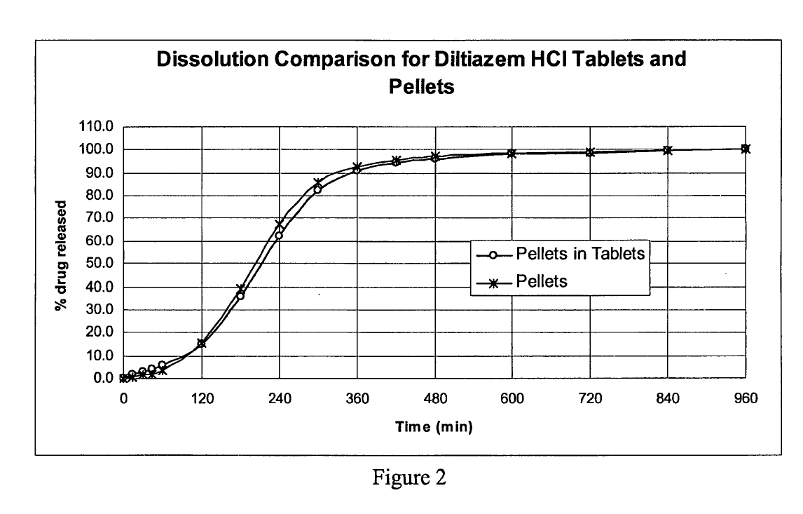

In one embodiment, the dissolution profile exhibited by the compressed pharmaceutical composition prepared by compressing a blend of compressible mixture and coated drug pellets is substantially the same as the dissolution profile of the coated drug pellets alone. As used herein, “substantially the same dissolution profile” means that the dissolution rate of the compressed pharmaceutical composition varies from the dissolution rate of the coated pellets by less than or equal to about 5 percent as determined at any point in the sharpest slope of the dissolution profile (e.g. between 720 and 1020 minutes in FIG. 1 and between 120 and 300 minutes in FIG. 2). The dissolution profile is substantially the same between the tablet and the coated pellets when tested according to an USP compendia method (e.g., 500 or 900 ml of purified water, USP aqueous buffer at pH 4.5, USP aqueous buffer at pH 6.8, USP aqueous buffer at pH 7.5, or 0.1N HCl at 37° C. in Apparatus 2 (USP 28, <711> Dissolution, paddle, 50 rpm or 100 rpm paddle speed).

In one embodiment, the difference between the dissolution rate of the compressed pharmaceutical composition varies from the dissolution rate of the coated pellets by less than or equal to about 12 percent wherein the difference is determined at any point in the sharpest slope of the dissolution profile (e.g. between 720 and 1020 minutes in FIG. 1 and between 120 and 300 minutes in FIG. 2), specifically less than or equal to about 10 percent, more specifically less than or equal to about 8 percent, and yet more specifically less than or equal to about 5 percent.

The drug pellets, coated drug pellets, and the compressed pharmaceutical compositions prepared from compressing a blend of pellets and compressible mixture can be characterized by their pharmacokinetic parameters. “Pharmacokinetic parameters” are parameters which describe the in vivo characteristics of the active agent over time, including for example the in vivo dissolution characteristics and plasma concentration of the active agent. By “Cₘₐₓ” is meant the measured concentration of the active agent in the plasma at the point of maximum concentration. By “C₂₄” is meant the concentration of the active agent in the plasma at about 24 hours. The term “Tₘₐₓ” refers to the time at which the concentration of the active agent in the plasma is the highest. “AUC” is the area under the curve of a graph of the concentration of the active agent (typically plasma concentration) vs. time, measured from one time to another.

In one embodiment, a compressed pharmaceutical composition (e.g. tablet) prepared by compressing a blend of compressible mixture and coated drug pellets, after oral administration thereof to a mammal, exhibits substantially the same bioavailability of the pharmaceutically active agent as the bioavailability of the pharmaceutically active agent achieved by the oral administration of the coated pellets in the absence of the compressible mixture. As disclosed herein, “substantially the same biovavailability” means that the bioavailability of the compressed pharmaceutical compositions exhibits an AUC, Tₘₐₓ, and Cₘₐₓ that varies by less than or equal to about 5 percent as compared to the coated pellets alone.

In one embodiment, a compressed pharmaceutical composition (e.g. tablet) prepared by compressing a blend of compressible mixture and coated drug pellets, exhibits an AUC, Tₘₐₓ, and Cₘₐₓ after oral administration thereof to a mammal that varies by less than or equal to about 15 percent from a corresponding dose of coated drug pellets dosed orally to a mammal in the absence of the compressible mixture. More specifically the AUC, Tₘₐₓ, and Cₘₐₓ of the compressed pharmaceutical composition varies by less than or equal to about 10 percent, yet more specifically less than or equal to about 7.5 percent.

In one embodiment, the compressible mixture is free of paraffin wax, microcrystalline wax, and/or microcrystalline cellulose.

The compressed pharmaceutical compositions provided herein can be formulated to be easily broken by hand using finger pressure without the presence of a score line. The compressed pharmaceutical composition can be broken into substantially uniform pieces without the aid of a score line. As used herein, “substantially uniform pieces” means that the weight of each piece is within about 10 percent of one another.

All references, including publications, patent applications, and patents, cited herein are hereby incorporated by reference to the same extent as if each reference were individually and specifically indicated to be incorporated by reference and were set forth in its entirety herein.

The following examples further illustrate the invention but, of course, should not be construed as in any way limiting its scope.

### EXAMPLE 1

Preparation of a Diltiazem Extended-Release Pellet:

Diltiazem extended-release pellets were prepared by coating 500 micrometer sugar spheres with diltiazem hydrochloride and hydroxypropyl cellulose to form a drug coated pellet. The drug coated pellet was then coated with a controlled-release coating, which was a combination of Eudragit RS and Eudragit RL in a 93.4: 6.6 ratio. This coating contributes to about 20% of the pellet weight. The average diameter size of the coated diltiazem pellets was about 900 micrometers.

Preparation of diltiazem tablets containing a carnauba wax/cross-linked sodium carboxymethylcellulose compressible mixture and diltiazem extended-release pellets:

The compressible mixture used to prepare the tablets include commercial grade powdered Carnauba wax and Ac-Di-Sol® available from FMC BioPolymer of Philadelphia, Pa. as the main components. The tablets were prepared by mixing all of the components of Table 1 except the magnesium stearate to form a uniform mixture followed by the addition of the magnesium stearate with mixing to form a final mixture. The final mixture was compressed into direct compression tablets using a Kilian LX Tablet press with an 0.75×0.45 inch oval-shaped tooling. Tablets were prepared for each of the following compression forces: 3, 4, 8, 10, and 14 kiloNewtons (kN), to produce tablets exhibiting a hardness of 4, 6, 7, 7, and 11 kiloponds (kp) respectively. No segregation was observed for the coated pellets and the compressible mixture. Such a result was especially surprising in view of the large size of the diltiazem pellets as compared to the remaining components which are powders.

TABLE 1

| mg/tablet | Ingredients | gram/batch |
|---|---|---|
| 400 | Diltiazem | 400 |
| | hydrochloride | |
| | Pellets | |
| 570 | Carnauba Wax | 570 |
| | (powder) | |
| 20 | AC-DI-SOL | 20 |
| 10 | Mg Stearate | 10 |
| 1000 | Total wt. | 1000 |

The compressed tablets and the diltiazem extended-release pellets were tested for dissolution according to USP 28 apparatus II using 900 milliliters 0.1 N HCl as the dissolution media at 37° C.±0.5° C. using a paddle speed of 100 rotations per minute (rpm). UV spectrometer was used to determine the amount of diltiazem dissolved at a given time point. The dissolution results are provided in Table 2 below as percentage of diltiazem released. The data is also illustrated graphically in FIG. 1. As can be seen from the comparison of the dissolution results, the compressed tablets exhibit a dissolution profile substantially identical to the free pellets. Such a result exhibits both the ability of the compressible mixture to protect the pellet coating while at the same time not delaying the dissolution of the diltiazem from the tablet. It is difficult to achieve a zero percent release as there will always be some minimal damage to the pellet or pellet coating for those pellets at the interface with the tableting tools. As indicated by the dissolution profiles in the Figure, such damage is minimized by the use of the compressible mixture.

TABLE 2

| | |
|---|---|
| Time | % Drug Released |

| (minutes) | Pellets in Tablets | Pellets |
|---|---|---|
| 0 | 0.0 | 0.0 |
| 15 | 0.7 | 0.1 |
| 30 | 0.9 | 0.1 |
| 45 | 1.3 | 0.5 |
| 60 | 1.0 | 0.2 |
| 120 | 1.5 | 0.2 |
| 180 | 1.9 | 0.2 |
| 240 | 2.2 | 0.1 |
| 300 | 3.0 | 0.3 |
| 360 | 3.2 | 0.0 |
| 420 | 3.6 | 0.4 |
| 480 | 3.9 | 2.3 |
| 540 | 3.9 | 2.3 |
| 600 | 4.8 | 1.5 |
| 720 | 10.8 | 8.6 |
| 840 | 43.5 | 40.6 |
| 900 | 63.8 | 60.6 |
| 960 | 79.7 | 76.9 |
| 1020 | 89.1 | 87.9 |
| 1080 | 93.7 | 93.0 |
| 1200 | 97.3 | 96.4 |
| 1320 | 98.5 | 97.9 |
| 1440 | 99.2 | 98.7 |
| 1560 | 99.1 | 99.3 |
| 1680 | 100.0 | 99.7 |
| 1800 | 100.0 | 100.0 |

### EXAMPLE 2

Preparation of an Additional Example of Diltiazem Extended-Release Pellet:

Diltiazem extended-release pellets were prepared according to Example 1, but the controlled-release coating composition contributes to about 10% of the pellet weight. The average diameter size of the coated diltiazem pellets was about 850 micrometers.

Preparation of diltiazem tablets containing a camauba wax/cross-linked sodium carboxymethylcellulose compressible mixture and diltiazem extended-release pellets:

The tablets were prepared with the components of Table 3, according to the procedure of Example 1. Tablets were prepared for each of the following compression forces: 4, 8, 12, and 21 kiloNewtons (kN), to produce tablets exhibiting a hardness of 7, 8, 10, and 10 kiloponds (kp) respectively. No segregation was observed for the coated pellets and the compressible mixture.

TABLE 3

| mg/tablet | Ingredients | gram/batch |
|---|---|---|
| 400 | Diltiazem | 400 |
| | hydrocloride Pellets | |
| 570 | Carnauba Wax | 570 |
| | (powder) | |
| 20 | AC-DI-SOL | 20 |
| 10 | Mg Stearate | 10 |
| 1000 | Total wt. | 1000 |

The compressed tablets and the diltiazem extended-release pellets were tested for dissolution according to the procedure of Example 1. The results of the dissolution analysis is provided in Table 4 below and illustrated graphically in FIG. 2. As can be seen from the data, the dissolution profile of the tablets prepared from the coated pellets and compressible mixture is substantially the same as the coated pellets themselves.

TABLE 4

% Drug Released

| Time | Pellets in Tablets | Pellets |
|---|---|---|
| 0 | 0.0 | 0.0 |
| 15 | 1.5 | 0.8 |
| 30 | 2.8 | 1.5 |
| 45 | 3.8 | 1.9 |
| 60 | 5.6 | 3.5 |
| 120 | 14.9 | 15.8 |
| 180 | 36.0 | 39.4 |
| 240 | 62.4 | 67.4 |
| 300 | 82.3 | 85.8 |
| 360 | 90.9 | 92.8 |
| 420 | 94.6 | 95.6 |
| 480 | 96.3 | 97.4 |
| 600 | 98.2 | 98.6 |
| 720 | 98.4 | 98.8 |
| 840 | 99.4 | 99.4 |
| 960 | 100.0 | 100.0 |

### EXAMPLE 3

Preparation Morphine Extended-Release Pellet:

Morphine extended-release pellets were prepared by coating a drug core containing morphine sulfate and hydroxypropyl methylcellulose to form a drug coated pellet. The drug coated pellet was coated with Eudragit L100 and ethylcellulose in a ratio of 77:23. The average diameter size of the morphine extended-release pellets was approximately 1400 micrometers.

Preparation of morphine extended-release tablets containing a carnauba cross-linked sodium carboxymethylcellulose compressible mixture and morphine extended-release pellets:

The tablets were prepared with the components of Table 5, according to the procedure of Example 1. No segregation was observed for the coated pellets and the compressible mixture.

TABLE 5

| mg/tablet | Ingredients | gram/batch |
|---|---|---|
| 400 | Morphine sulfate | 400 |
| | Pellets | |
| 570 | Carnauba Wax | 570 |
| | (powder) | |
| 20 | AC-DI-SOL | 20 |
| 10 | Mg Stearate | 10 |
| 1000 | Total wt. | 1000 |

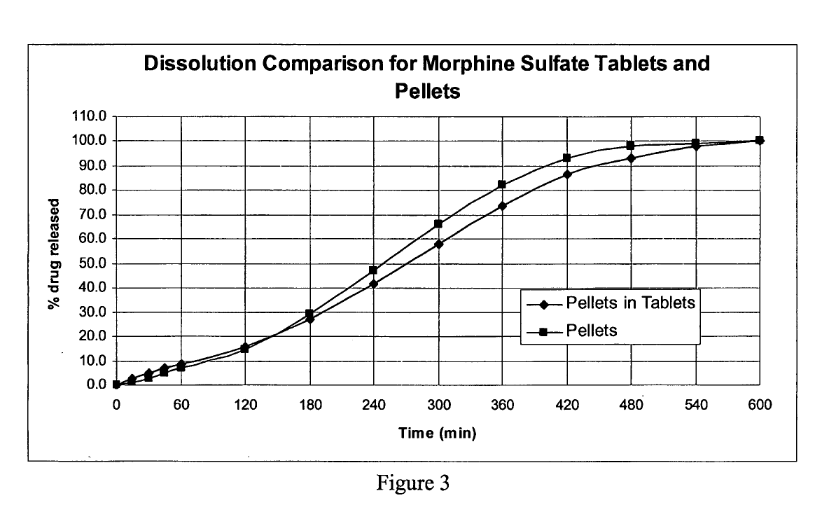

The compressed tablets and the morphine extended-release pellets were tested for dissolution according to the procedure of Example 1, but the dissolution medium used was 900 ml 0.1 N HCl for two (2) hours and then in pH 6.8 phosphate buffer for eight (8) hours. The results of the dissolution analysis is provided in Table 6 below and illustrated graphically in FIG. 3. As can be seen from the data, the dissolution profile of the tablets prepared from the coated pellets and compressible mixture is slightly slower as compared to the coated pellets alone, but with the same overall endpoint. As indicated, the result was minimal damage to the pellets or the coatings on the pellets and therefore no increase in the dissolution.

TABLE 6

% Drug Released

| Time | Pellets in Tablets | Pellets |
|---|---|---|
| 0 | 0.0 | 0.0 |
| 15 | 2.8 | 1.2 |
| 30 | 4.6 | 2.6 |
| 45 | 7.1 | 4.9 |
| 60 | 8.9 | 6.8 |
| 120 | 15.9 | 14.8 |
| 180 | 27.0 | 29.0 |
| 240 | 41.7 | 47.2 |
| 300 | 57.9 | 66.1 |
| 360 | 73.5 | 82.5 |
| 420 | 86.8 | 93.3 |
| 480 | 93.0 | 97.9 |
| 540 | 97.9 | 99.3 |
| 600 | 100.0 | 100.0 |

### EXAMPLE 4

Preparation of an Amphetamine Extended-Release Pellet:

Amphetamine extended-release pellets were prepared using a combination of Eudragit RS and Eudragit RL in a 90:10 ratio. The resulting extended-release amphetamine pellets had an average diameter size of 640 micrometers.

Preparation of amphetamine extended-release tablets containing a carnauba wax/cross-linked sodium carboxymethylcellulose compressible mixture and amphetamine extended-release pellets:

The tablets were prepared with the components of Table 7, according to the procedure of Example 1 above. No segregation was observed for the coated pellets and the compressible mixture.

TABLE 7

| mg/tablet | Ingredients | gram/batch |
|---|---|---|
| 400 | Amphetamine | 400 |
| | Pellets | |
| 570 | Carnauba Wax | 570 |
| | (powder) | |
| 20 | AC-DI-SOL | 20 |
| 10 | Mg Stearate | 10 |
| 1000 | Total wt. | 1000 |

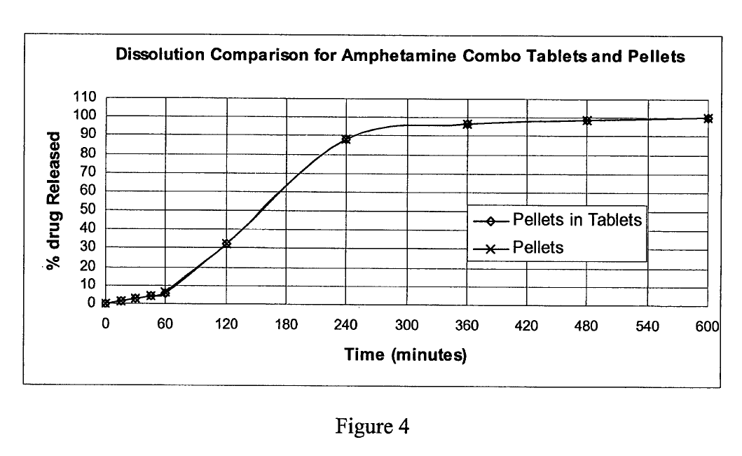

The compressed tablets and the amphetamine extended-release pellets were tested for dissolution according to the procedure of Example 1. The dissolution samples were analyzed by HPLC. The results of the dissolution analysis is provided in Table 8 below and illustrated graphically in FIG. 4. As can be seen from the data, the dissolution profile of the tablets prepared from the coated pellets and compressible mixture is substantially the same as the coated pellets themselves.

TABLE 8

% Drug Released

| Time | Pellets in Tablets | Pellets |
|---|---|---|
| 0 | 0.0 | 0.0 |
| 15.0 | 1.1 | 1.1 |
| 30.0 | 2.6 | 2.8 |
| 45.0 | 4.1 | 4.2 |
| 60.0 | 5.7 | 6.1 |
| 120.0 | 32.4 | 32.7 |
| 240.0 | 88.1 | 88.0 |
| 360.0 | 96.5 | 96.4 |
| 480.0 | 98.9 | 98.6 |
| 600.0 | 100.0 | 100.0 |

### EXAMPLE 5

Preparation of Diltiazem Tablets Containing a Camauba Wax/Microcrystalline Cellulose/Cross-Linked Sodium Carboxymethylcellulose Compressible Mixture and Diltiazem Extended-Release Pellets:

The materials used to prepare the tablets include commercial grade Avicel PH102, Carnauba wax and Ac-Di-Sol(D available from FMC BioPolymer of Philadelphia, Pa. The tablets were prepared by mixing all of the components of Table 9 except the magnesium stearate to form a uniform mixture followed by the addition of the magnesium stearate with mixing to form a final mixture. The final mixture was compressed into direct compression tablets using a Kilian LX Tablet press with an 0.497×0.272 inch oval-shaped tooling. Tablets were prepared for each of the following compression forces: 8 and 10 kN, to produce tablets exhibiting a hardness of about 8 to 12 kp, respectively. No segregation was observed for the coated pellets and the compressible mixture.

TABLE 9

| mg/Tablet | Ingredients | gram/batch |
|---|---|---|
| 133 | Diltiazem Pellets | 400 |
| 157 | Carnauba Wax | 470 |
| | (powder) | |
| 33 | Avicel PH102 | 100 |
| 7 | AC-DI-Sil | 20 |
| 3 | Mg Stearate | 10 |
| 333 | Total wt. | 1000 |

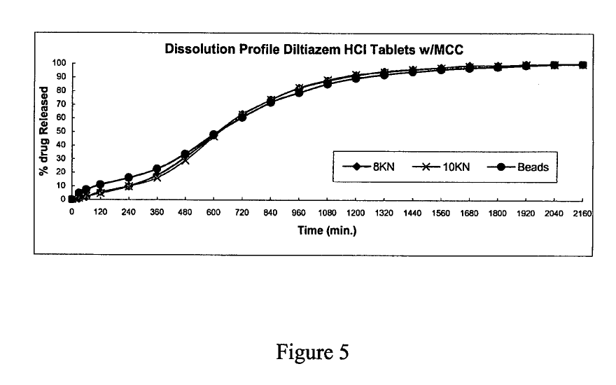

The compressed tablets for both compression forces and the diliazem extended-release pellets were tested for dissolution according to USP 28 apparatus II using 900 milliliters 0.1 N HCl as the dissolution media at 37° C.±0.5° C. using a paddle speed of 100 rotations per minute (rpm). Analysis of the dissolution samples were performed according to Example 1. The dissolution results are provided in table 10 below as percentage of diltiazem released. The data is also illustrated graphically in FIG. 5. As can be seen from the comparison of the dissolution results, both of the compressed tablets exhibit a dissolution profile substantially identical to the free pellets. The particular combination of the camauba wax powder, microcrystalline cellulose and disintegrant provides a compressible mixture capable of forming strong tablets (as evidenced by the hardness results) by compression while at the same time providing enough protection for the coated beads to maintain the integrity of the coating (as evidenced by the dissolution results). Furthermore, no segregation of the compressible mixture from the pellets was observed during the tableting process.

TABLE 10

| | % Drug Released | | |
|---|---|---|---|
| Time | Pellets in Tablets | Pellets in Tablets | |
| (minutes) | 8 KN | 10 KN | Pellets |
| 0 | 0.0 | 0.0 | 0.0 |
| 30 | 0.8 | 1.1 | 4.7 |
| 60 | 2.0 | 2.5 | 7.3 |
| 120 | 5.6 | 4.8 | 11.1 |
| 240 | 10.1 | 9.7 | 16.1 |
| 360 | 18.0 | 16.1 | 22.6 |
| 480 | 31.1 | 28.8 | 33.8 |
| 600 | 47.6 | 46.6 | 48.0 |
| 720 | 63.0 | 62.7 | 60.6 |
| 840 | 73.7 | 73.7 | 71.9 |
| 960 | 82.3 | 82.5 | 78.8 |
| 1080 | 87.6 | 88.2 | 85.4 |
| 1200 | 91.7 | 92.2 | 89.5 |
| 1320 | 94.4 | 93.9 | 92.1 |
| 1440 | 95.9 | 95.7 | 94.3 |
| 1560 | 97.3 | 97.0 | 95.9 |
| 1680 | 98.9 | 98.1 | 97.1 |
| 1800 | 98.9 | 98.7 | 98.0 |
| 1920 | 99.6 | 99.6 | 99.0 |
| 2040 | 100 | 99.8 | 99.8 |
| 2160 | 100 | 100 | 100 |

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention as used herein, the terms weight percent, weight percent, percent by weight, etc. are equivalent and interchangeable.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. All ranges disclosed herein are inclusive and combinable.

What is claimed is:

1. A compressed pharmaceutical composition, comprising:
a plurality of coated pellets, wherein the coated pellets comprise a pharmaceutically active agent or salt, solvate, hydrate, or polymorph thereof; and
a compressible mixture, wherein the compressible mixture comprises i) a non-water soluble waxy filler and ii) a disintegrant, and wherein the compressible mixture is in powder form, with the proviso that the compressible mixture is not prepared by extrusion, spheronization, high shear mixing, melt blending, melt pelletization, freeze-drying, or a combination thereof, and
wherein the compressed composition exhibits an in vitro dissolution profile according to a USP compendia method that is substantially the same as the dissolution of the coated pellets in the absence of the compressible mixture.

2. The compressed pharmaceutical composition of claim 1, wherein, after oral administration thereof to a mammal, the bioavailability of the pharmaceutically active agent from the compressed composition is substantially the same as the bioavailability of the pharmaceutically active agent achieved by the administration of the coated pellets in the absence of the compressible mixture.

3. The compressed pharmaceutical composition of claim 1, wherein the USP compendia method is USP 28, <711> Dissolution, Apparatus 2, paddle, paddle speed of 100 rpm and 900 ml of 0.1 N HCl as a dissolution medium at 37° C.

4. The compressed pharmaceutical composition of claim 1, wherein the waxy filler is carnauba wax, vegetable wax, fruit wax, microcrystalline wax, bees wax, hydrocarbon wax, paraffin wax, cetyl esters wax, non-ionic emulsifying wax, anionic emulsifying wax, candelilla wax, stearyl alcohol, cetyl alcohol, cetostearyl alcohol, or combinations thereof; and
the disintegrant is cross-linked sodium carboxymethylcellulose, crosslinked homopolymer of N-vinyl-2-pyrrolidone, modified starches, sodium carboxymethyl starch, sodium starch glycolate, alginates, starch, pregelatinized starch, or combinations thereof.

5. The compressed pharmaceutical composition of claim 1, wherein the waxy filler is powdered carnauba wax and the disintegrant is cross-linked sodium carboxymethylcellulose.

6. The compressed pharmaceutical composition of claim 1, wherein the amount of waxy filler is about 25 to about 75 weight percent based on the total weight of the compressed pharmaceutical composition.

7. The compressed pharmaceutical composition of claim 1, wherein the amount of disintegrant is about 1 to about 5 weight percent based on the total weight of the compressed pharmaceutical composition.

8. The compressed pharmaceutical composition of claim 1, further comprising an additional excipient, an additional disintegrant, a coating disposed on the surface of the compressed composition, or combinations thereof.

9. The compressed pharmaceutical composition of claim 8, wherein the additional excipient is a binder, a lubricant, a glidant, a compression aid, a colorant, a preservative, a flavor, or combinations thereof.

10. The compressed pharmaceutical composition of claim 1, wherein the compressible mixture further comprises a cellulose filler.

11. The compressed pharmaceutical composition of claim 1, wherein the compressible mixture further comprises microcrystalline cellulose in a weight ratio to the waxy filler of about 5:95 to about 40:60 (w/w).

12. The compressed pharmaceutical composition of claim 1, wherein the pharmaceutically active agent or salt thereof is an alpha-2 adrenergic agent, an analgesic, an angiotensin-converting enzyme (ACE) inhibitor, an antianxiety agent, an antiarrhythmic, an antibacterial, an antibiotic, an antidepressant, an antidiabetic, an antiemetic, an antiepileptic, an antifungal, an antihelminthic, an antihistamine, an antihyperlipidemic, an antihypertensive agent, an antiinfective, an antimalarial, an antimicrobial, an antimigraine agent, an antimuscarinic agent, an antineoplastic agent, an antiprotozoal agent, an antipsychotic agent, an antispasmodic, an antiviral agent, an attention-deficit hyperactivity disorder (ADHD) agent, a β-blocker, a calcium channel blocker, a chemotherapeutic agent, a cholinesterase inhibitor, a Cox-2 inhibitor, a decongestant, a diuretic, a histamine-2 receptor antagonist, a hypnotic, a hypotensive agent, an immunosuppressant, a lipotropic, a neuroleptic, an opioid analgesic, a peripheral vasodilator/vasoconstrictor, a sedative, a serotonin receptor agonist, or pharmaceutically acceptable combinations thereof.

13. The compressed pharmaceutical composition of claim 1, wherein the pharmaceutically active agent is amphetamine, dextroamphetamine, diltiazem, fluvastatin, hydromorphone, morphine, oxybutynin, oxycodone, paroxetine, propranolol, tolterodine, venlafaxine, or the pharmaceutically acceptable salt, solvate, hydrate, or polymorph thereof.

14. The compressed pharmaceutical composition of claim 1, wherein the coated pellets comprise a coating prepared from alkyl cellulose, methyl cellulose, ethyl cellulose, hydroxypropyl cellulose, hydroxypropyl methyl cellulose, hydroxybutyl methyl cellulose, cellulose acetate, cellulose propionate, cellulose acetate propionate, cellulose acetate butyrate, cellulose acetate phthalate, carboxymethyl cellulose, cellulose triacetate, cellulose sulphate sodium salt, poly(methyl methacrylate), poly (ethyl methacrylate), poly (butyl methacrylate), poly (isobutyl methacrylate), poly (hexyl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene) high density, (poly propylene), poly (ethylene glycol), poly (ethylene oxide), poly (ethylene terephthalate), poly(vinyl alcohol), poly(vinyl isobutyl ether), poly(vinyl acetate), poly (vinyl chloride), polyvinyl pyrrolidone, or combinations thereof; and optionally further comprising a plasticizer.

15. The compressed pharmaceutical composition of claim 1, wherein the coated pellets have an average diameter size of about 100 to about 3000 micrometers.

16. The compressed pharmaceutical composition of claim 1, wherein the compressible mixture comprises particles having an average diameter size of about 0.1 to about 125 micrometers.

17. The compressed pharmaceutical composition of claim 1, wherein the compressed pharmaceutical composition has a hardness value of at least about 8 kiloponds.

18. A method of treating a mammal in need of a therapeutic amount of a pharmaceutical agent comprising administering orally the compressed pharmaceutical composition of claim 1.

19. A process of making a compressed pharmaceutical composition, comprising:
mixing a powdered, waxy filler and a disintegrant to form a compressible mixture, wherein the waxy filler is non-water soluble;
mixing the compressible mixture with a plurality of coated pellets to form a pellet mixture, wherein the coated pellets comprise a pharmaceutically active agent or salt, solvate, hydrate, or polymorph thereof; and
compressing the pellet mixture with conventional direct compression equipment, wherein the compression force is not more than about 25 kiloNewtons.

20. A direct compression compressible mixture for forming compressed pharmaceutical compositions, comprising:
a mixture of a powdered waxy filler and a disintegrant, optionally further comprising a cellulose filler, a binder, a lubricant, a glidant, a compression aid, a colorant, a preservative, a flavor, or combinations thereof,
wherein the mixture has an average particle diameter of about 0.1 to about 125 micrometers, with the proviso that the compressible mixture is not prepared by extrusion, spheronization, high shear mixing, melt blending, melt pelletization, freeze-drying, or a combination thereof; and
wherein the compressible mixture is suitable for directly compressing mixtures of the compressible mixture and coated drug pellets with substantially no damage to the coated drug pellets.

21. The compressible mixture of claim 20, wherein the powdered waxy filler is camauba wax, vegetable wax, fruit wax, microcrystalline wax, bees wax, hydrocarbon wax, paraffin wax, cetyl esters wax, non-ionic emulsifying wax, anionic emulsifying wax, candelilla wax, stearyl alcohol, cetyl alcohol, cetostearyl alcohol, polyethylene or combinations thereof; and
wherein the disintegrant is cross-linked sodium carboxymethylcellulose, crosslinked homopolymer of N-vinyl-2-pyrrolidone, modified starches, sodium carboxymethyl starch, sodium starch glycolate, alginates, starch, pregelatinized starch, and combinations thereof.
